(19) United States
(12) Patent Application Publication
(10) Pub. No.: US 2025/0073427 A1
(43) Pub. Date: Mar. 6, 2025

(54) CATHETER SECUREMENT DEVICE, KIT, AND METHOD OF USE THEREOF

(71) Applicant: 3M INNOVATIVE PROPERTIES COMPANY, St. Paul, MN (US)

(72) Inventor: Ganesh P. D. Kannan, Bangalore (IN)

(21) Appl. No.: 18/293,119

(22) PCT Filed: Jul. 27, 2022
(86) PCT No.: PCT/IB2022/056936

§ 371 Date: Jan. 29, 2024

Related U.S. Application Data
(60) Provisional application No. 63/229,738, filed on Aug. 5, 2021.

(51) Int. Cl. A61M 25/02

(52) U.S. Cl. CPC A61M 25/02; A61M 2025/028

(57) ABSTRACT

A catheter securement device (100) for use with a catheter assembly (10) includes abase (110) configured to be removably secured to a skin (20) of a user (22), a plurality of lock tabs (140) fixedly attached to the base (110) and extending from a first major surface (112) of the base (110), and a housing (160) configured to be placed on the first major surface (112) of the base (110). The housing (160) and the first major surface (112) of the base (110) at least partially enclose the catheter assembly (10) there-between. The housing (160) includes a plurality of lock handles (162). Each lock handle (162) from the plurality of lock handles (162) defines an aperture (164) therethrough. Each lock tab (140) is configured to be received through the aperture (164) of a corresponding lock handle (162) from the plurality of lock handles (164). Furthermore, each lock tab (140) is configured to be detachably attached to the first major surface (112) of the base (110), such that the housing (160) is removably secured to the base (110).

### TECHNICAL FIELD

The present disclosure relates generally to a catheter securement device, a kit including the catheter securement device, and a method for use with a catheter assembly.

### BACKGROUND

Catheter assemblies may be used for various purposes, such as feeding, air supply, and/or liquid removal. In some cases, catheter assemblies may be used to administer medications and fluids to a user. In many instances, the catheter assemblies may need to be secured to a skin of the user to prevent movement of the catheter assemblies. For example, the catheter assemblies may need to be secured to the skin of the user to prevent the catheter assemblies from being pulled out, or otherwise moved in ways that may adversely affect functioning of the catheter assemblies.

The catheter assemblies are generally secured to the skin of the user using conventional catheter securement units, such as tapes, patches, and sutures. However, the conventional catheter securement units may not properly secure the catheter assemblies to the skin. Specifically, the conventional catheter securement units may not properly stabilize the catheter assemblies, thereby allowing undesirable movement of the catheter assemblies during use. Therefore, in some cases, use of the conventional catheter securement units may result in various complications, such as dislodgment and accidental removal of the catheter assemblies from the skin of the user, phlebitis, extravasation/infiltration, leakage, occlusion, and bloodstream infections. Furthermore, the conventional catheter securement units may not provide proper coverage of an injection site, which may result in infection at the injection site. Moreover, use of sutures to secure the catheter assemblies to the skin may lead to suture related infections and complications.

### SUMMARY

Generally, the present disclosure relates to a catheter securement device for use with a catheter assembly. The present disclosure further relates to a kit including the catheter assembly and the catheter securement device, and a method for use with the catheter assembly.

In a first aspect, the present disclosure provides a catheter securement device for use with a catheter assembly. The catheter securement device includes a base configured to be removably secured to a skin of a user. The base includes a first major surface and a second major surface opposite to the first major surface. The first major surface is configured to at least partially receive the catheter assembly thereon. The second major surface is configured to face the skin of the user. The catheter securement device further includes a plurality of lock tabs fixedly attached to the base and extending from the first major surface of the base. The lock tabs are spaced apart from each other. The catheter securement device further includes a housing configured to be placed on the first major surface of the base, such that the housing and the first major surface of the base at least partially enclose the catheter assembly therebetween. The housing includes a plurality of lock handles. Each lock handle from the plurality of lock handles defines an aperture therethrough. Each lock tab is configured to be received through the aperture of a corresponding lock handle from the plurality of lock handles. Upon being received through the aperture of the corresponding lock handle, each lock tab is configured to be detachably attached to the first major surface of the base, such that the housing is removably secured to the base.

In a second aspect, the present disclosure provides a kit including a catheter assembly and a catheter securement device for securing the catheter assembly to a skin of a user. The catheter securement device includes a base configured to be removably secured to the skin of the user. The base includes a first major surface and a second major surface opposite to the first major surface. The first major surface is configured to at least partially receive the catheter assembly thereon. The second major surface is configured to face the skin of the user. The catheter securement device further includes a plurality of lock tabs fixedly attached to the base and extending from the first major surface of the base. The lock tabs are spaced apart from each other. The catheter securement device further includes a housing configured to be placed on the first major surface of the base, such that the housing and the first major surface of the base at least partially enclose the catheter assembly therebetween. The housing includes a plurality of lock handles. Each lock handle from the plurality of lock handles defines an aperture therethrough. Each lock tab is configured to be received through the aperture of a corresponding lock handle from the plurality of lock handles. Upon being received through the aperture of the corresponding lock handle, each lock tab is configured to be detachably attached to the first major surface of the base, such that the housing is removably secured to the base.

In a third aspect, the present disclosure provides a method for use with a catheter assembly. The method includes providing a base including a first major surface and a second major surface opposite to the first major surface. The method further includes providing a plurality of lock tabs fixedly attached to the base and extending from the first major surface of the base. The method further includes providing a housing including a plurality of lock handles. Each lock handle from the plurality of lock handles defines an aperture therethrough. The method further includes disposing at least partially the catheter assembly on a skin of a user. The method further includes placing the base underneath the catheter assembly, such that the first major surface of the base at least partially receives the catheter assembly thereon and the second major surface faces the skin of the user. The method further includes detachably securing the base to the skin of the user, such that the second major surface faces the skin of the user. The method further includes slidably receiving the housing on the plurality lock tabs, such that each lock tab is received through the aperture of a corresponding lock handle from the plurality of lock handles. The method further includes disposing the housing on the first major surface of the base, such that the catheter assembly is at least partially enclosed between the first major surface of the base and the housing. The method further includes detachably attaching the plurality of lock tabs to the first major surface of the base, such that the housing is detachably secured to the base.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 illustrates a schematic perspective view of a catheter assembly according to an embodiment of the present disclosure;

FIG. 2 illustrates a schematic partially exploded perspective view of a catheter securement device according to an embodiment of the present disclosure;

FIG. 3 illustrates a schematic cross-sectional view of a portion of the catheter securement device according to an embodiment of the present disclosure;

FIG. 4A illustrates a schematic perspective view of the catheter securement device according to an embodiment of the present disclosure;

FIG. 4B illustrates a schematic cross-sectional view of a portion of the catheter securement device taken along a line 1-1 of FIG. 4A according to an embodiment of the present disclosure;

FIG. 4C illustrates a schematic cross-sectional view of a portion of the catheter securement device taken along a line 2-2 of FIG. 4B according to an embodiment of the present disclosure;

FIG. 5A illustrates a schematic front perspective view of the catheter securement device according to an embodiment of the present disclosure;

FIG. 5B illustrates a schematic top view of the catheter securement device according to an embodiment of the present disclosure;

FIG. 5C illustrates a schematic rear perspective view of the catheter securement device according to an embodiment of the present disclosure;

FIG. 6 illustrates a schematic block diagram of a kit according to an embodiment of the present disclosure; and

FIG. 7 illustrates a flowchart depicting various steps of a method for use with the catheter assembly according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials comprises less than about 10 weight % of each of the first and second materials.

As used herein, “at least one of A and B” should be understood to mean “only A, only B, or both A and B”.

Unless specified or limited otherwise, the terms “attached,” “connected,” and variations thereof, are used broadly and encompass both direct and indirect attachments, connections, and couplings.

As used herein, the terms “layer,” “sheet,” and “dressing,” or variations thereof, are used to describe an article having a thickness that is small relative to its length and width.

As used herein, the term “polymer” refers to both materials prepared from one monomer such as a homopolymer or to materials prepared from two or more monomers such as a copolymer, terpolymer, or the like. Likewise, the term “polymerize” refers to the process of making a polymeric material that can be a homopolymer, copolymer, terpolymer, or the like. The term “copolymer” refers to a polymeric material prepared from at least two different monomers.

The present disclosure provides a catheter securement device for use with a catheter assembly. The catheter securement device includes a base configured to be removably secured to a skin of a user. The base includes a first major surface and a second major surface opposite to the first major surface. The first major surface is configured to at least partially receive the catheter assembly thereon. The second major surface is configured to face the skin of the user. The catheter securement device further includes a plurality of lock tabs fixedly attached to the base and extending from the first major surface of the base. The lock tabs are spaced apart from each other. The catheter securement device further includes a housing configured to be placed on the first major surface of the base, such that the housing and the first major surface of the base at least partially enclose the catheter assembly therebetween. The housing includes a plurality of lock handles. Each lock handle from the plurality of lock handles defines an aperture therethrough. Each lock tab is configured to be received through the aperture of a corresponding lock handle from the plurality of lock handles. Upon being received through the aperture of the corresponding lock handle, each lock tab is configured to be detachably attached to the first major surface of the base, such that the housing is removably secured to the base.

Conventional catheter securement units, such as tapes, patches, and sutures may not properly secure the catheter assemblies to the skin of a user. Specifically, the conventional catheter securement units may not properly stabilize the catheter assemblies, thereby allowing undesirable movement of the catheter assemblies during use. Therefore, in some cases, use of the conventional catheter securement units may result in various complications, such as dislodgment and accidental removal of the catheter assemblies from the skin of the user, phlebitis, extravasation, occlusion/infiltration, leakage, and bloodstream infections. Furthermore, the conventional catheter securement units may not provide proper coverage of an injection site, which may result in infection at the injection site. Moreover, use of sutures to secure the catheter assemblies to the skin may lead to suture related infections and complications.

The catheter securement device of the present disclosure may firmly secure the catheter assembly to the skin of the user. Specifically, the catheter securement device may firmly secure various components (such as a catheter hub, one or more lumens, and a cannula) of the catheter assembly to the skin of the user.

The base may be removably secured to the skin of the user. Each lock tab may secure the corresponding lock handle of the housing to the base and inhibit movement of the housing relative to the base. Moreover, the housing of the catheter securement device may engage with the catheter assembly and inhibit movement of the catheter assembly relative to the housing. In some cases, the housing of the catheter securement device may include one or more stabilizer lugs to engage with the catheter assembly and inhibit movement of the catheter assembly relative to the housing.

Thus, the catheter securement device may prevent dislodgement and accidental removal of the catheter assembly from the skin of the user. Further, the catheter securement device may prevent various complications, such as phlebitis, extravasation/infiltration, leakage, occlusion, and bloodstream infections that may otherwise occur due to movement of the catheter assembly.

In some cases, the catheter securement device may include a closure tab to at least partially cover the injection site. The closure tab may protect the injection site from micro-organisms and prevent infections. Moreover, in some cases, the base of the catheter securement device may include a stretch-release adhesive. The stretch-release adhesive may facilitate removal of the base from the skin of the user. In other words, the base may be easily removed from the skin of the user without causing discomfort to the user.

Therefore, the catheter securement device may firmly secure the catheter assembly to the skin of the user, may inhibit undesirable movement of the catheter assembly, may prevent infection at the injection site, and may facilitate removal of the catheter securement device after use.

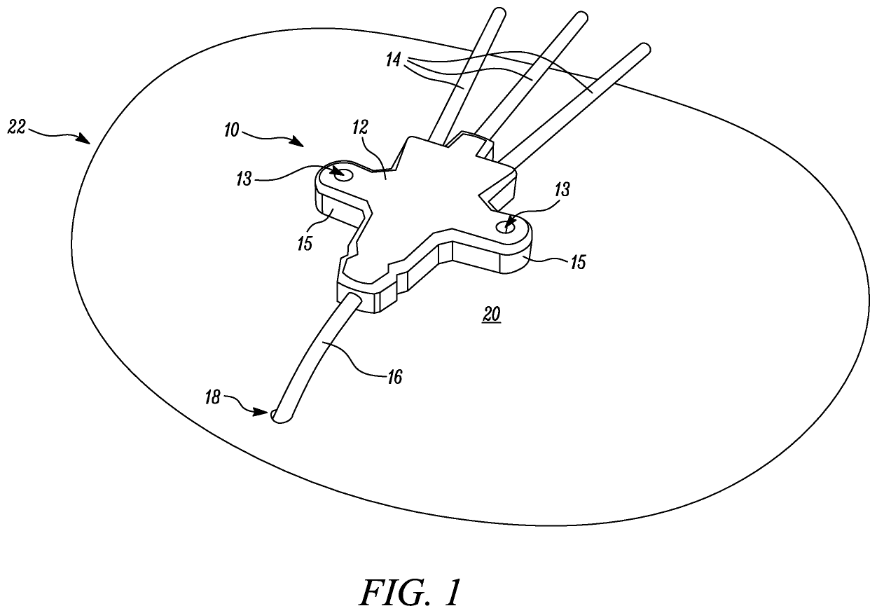

Referring now to Figures, FIG. 1 illustrates a schematic perspective view of a catheter assembly 10 according to an embodiment of the present disclosure. In FIG. 1, the catheter assembly 10 is partially disposed on a skin 20 of a user 22. In some cases, the user 22 may be a patient undergoing an intravenous therapy.

In the illustrated embodiment of FIG. 1, the catheter assembly 10 includes a catheter hub 12, one or more lumens 14, and a cannula 16.

In the illustrated embodiment of FIG. 1, the one or more lumens 14 are at least partially received within the catheter hub 12. The one or more lumens 14 are partially shown in FIG. 1. In the illustrated embodiment of FIG. 1, the one or lumens 14 include three lumens 14. In other words, in the illustrated embodiment of FIG. 1, the catheter assembly 10 is a triple lumen catheter assembly. However, a number of the one or more lumens 14 may vary based on desired application and/or infusion attributes. The one or more lumens 14 are fluidly connected with the cannula 16.

In the illustrated embodiment of FIG. 1, the cannula 16 is at least partially received within the catheter hub 12. In FIG. 1, the cannula 16 of the catheter assembly 10 is inserted into an injection site 18 on the skin 20 of the user 22. In other words, in FIG. 1, the cannula 16 is inserted through the skin 20 of the user 22 at the injection site 18.

In the illustrated embodiment of FIG. 1, the catheter hub 12 includes a pair of opposing wings 15. Furthermore, in the illustrated embodiment of FIG. 1, the catheter hub 12 includes one or more holes 13. Specifically, in the illustrated embodiment of FIG. 1, the catheter hub 12 includes a corresponding hole 13 on each of the pair of opposing wings 15.

In some embodiments, the catheter assembly 10 may include a central venous catheter (CVC). The CVC may be referred to as a central line, a central venous line, or a central venous access catheter. The CVC may be used to access large, centrally located veins, which is often required for critically ill patients, for patients requiring prolonged intravenous therapies for reliable vascular access, and to administer fluids that may harm smaller peripheral veins. Therefore, in some embodiments, at least a portion of the cannula 16 may be inserted into veins located at a neck (e.g., an internal jugular vein), into veins located at a chest (e.g., a subclavian vein or axillary vein), or into veins located at a groin (e.g., a femoral vein) of the user 22. In some embodiments, the catheter assembly 10 may include a peripherally inserted central catheter (PICC). The PICC may be suitable for insertion into veins located at an arm of the user 22. However, the catheter assembly 10 may include any suitable type of catheter, which may be selected based on desired application attributes.

Thus, the catheter assembly 10 may be used to administer an intravenous therapy (e.g., medication or fluids for parenteral nutrition), to obtain blood for analysis, and/or to provide an access point for blood-based treatments, such as dialysis or apheresis. The catheter assembly 10 may further be used obtain blood tests (e.g., a “central venous oxygen saturation”), administer fluid or blood products for a large volume resuscitation, and/or measure a central venous pressure.

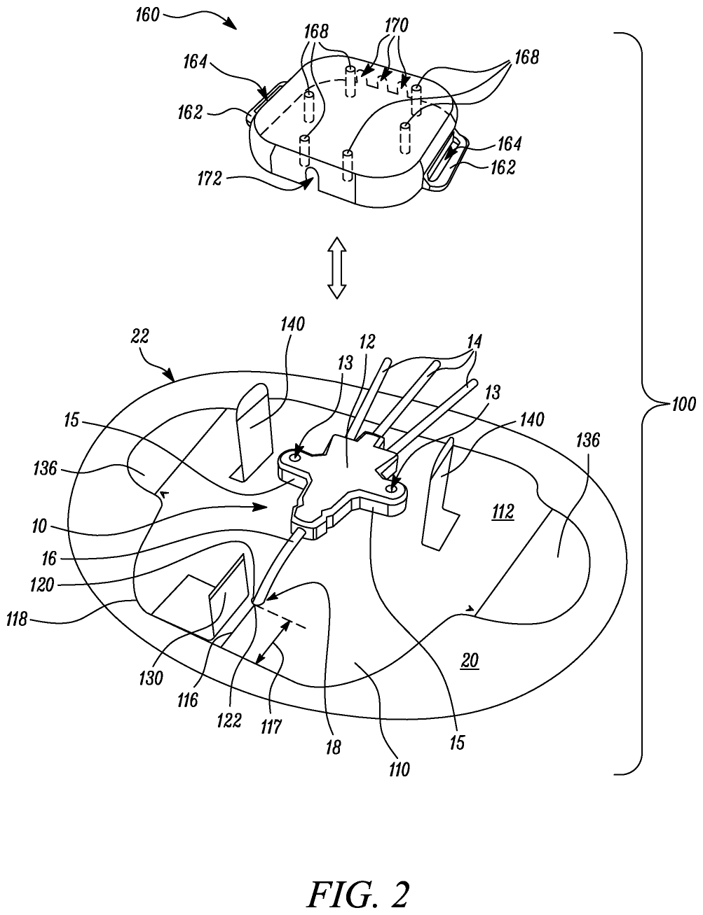

FIG. 2 illustrates a partially exploded perspective view of a catheter securement device 100 for use with the catheter assembly 10.

The catheter securement device 100 includes a base 110 configured to be removably secured to the skin 20 of the user 22. In other words, the base 110 may be removably attached to and detached from the skin 20 of the user 22 when desired. In some embodiments, the base 110 may be removably secured to the skin 20 of the user 22 by a suitable adhesive, such as a pressure-sensitive adhesive.

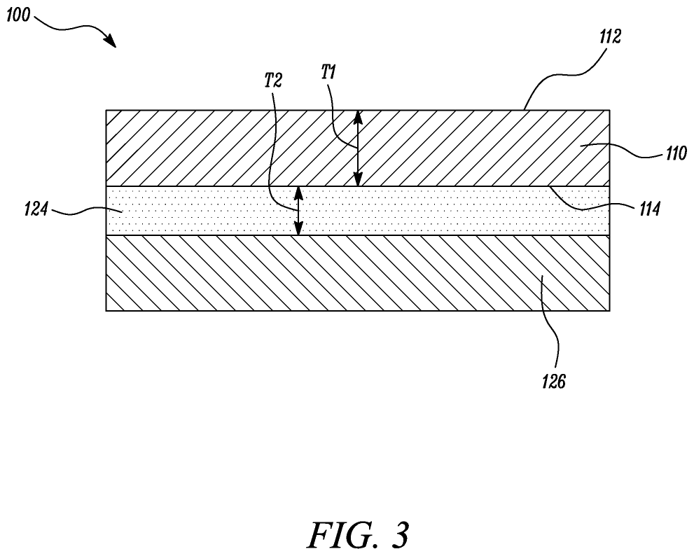

The base 110 includes a first major surface 112 and a second major surface 114 (shown in FIG. 3) opposite to the first major surface 112. The first major surface 112 is configured to at least partially receive the catheter assembly 10 thereon. Moreover, the second major surface 114 is configured to face the skin 20 of the user 22. In the illustrated embodiment of FIG. 2, the catheter hub 12 of the catheter assembly 10 is substantially received on the first major surface 112 of the base 110.

The base 110 may be made of a stretchable material that may conform to undulated surfaces (e.g., the skin 20 of the user 22). Therefore, in some embodiments, the base 110 may be configured to exhibit high elasticity. In some embodiments, a modulus of elasticity of the base 110 may be at least about 2,500 pounds per square inch (psi) or at least about 3,000 psi. In some embodiments, the modulus of elasticity of the base 110 may be less than about 50,000 psi. In some embodiments, the modulus of elasticity of the base 110 may be between about 5,000 psi and about 30,000 psi. In some embodiments, the base 110 may have a high tensile strength, a low elastic recovery, and a high elongation at break.

In some embodiments, the base 110 includes at least one of a polymeric film, a polymeric foam, a polymeric hydrocolloid, and a polymeric alginate. In some embodiments, the base 110 may include polyolefins, such as polyethylene, including high density polyethylene, low density polyethylene, linear low density polyethylene, and linear ultra-low density polyethylene, polypropylene, and polybutylenes; vinyl copolymers, such as polyvinyl chlorides, both plasticized and unplasticized, and polyvinyl acetates; olefinic copolymers, such as ethylene/methacrylate copolymers, ethylene/vinyl acetate copolymers, acrylonitrile-butadiene-styrene copolymers, and ethylene propylene copolymers; acrylic polymers and copolymer; and combinations thereof. In some embodiments, the base 110 may be in a form of single or multi-layer films, non-woven films, porous films, foam-like films, and combinations thereof. In some embodiments, the base 110 may be fabricated using any suitable method of film forming, such as extrusion, co-extrusion, solvent casting, foaming, non-woven technology, and the like.

In some embodiments, the base 110 may be a passive dressing. In other words, in some embodiments, the base 110 may be non-occlusive. However, in some embodiments, the base 110 may be an interactive dressing. In other words, in some embodiments, the base 110 may be semi-occlusive or occlusive. Therefore, in some embodiments, the base 110 may act as a barrier against penetration of bacteria. In some cases, the base 110 may maintain hydration of an area of the skin 20 at which the base 110 is removably secured, and may reduce infection of the area of the skin 20 at which the base 110 is removably secured.

In the illustrated embodiment of the FIG. 2, the base 110 defines a slit 116 extending from aperimeter 118 of the base 110 and a through hole 120 at an end 122 of the slit 116 distal to the perimeter 118 of the base 110. In the illustrated embodiment of FIG. 2, the slit 116 extends from the perimeter 118 to the through hole 120. In other words, in the illustrated embodiment of FIG. 2, the slit 116 extends between the perimeter 118 and the through hole 120. The slit 116 and the through hole 120 may define a keyhole-shaped through opening of the base 110. In the illustrated embodiment of FIG. 2, the through hole 120 is configured to receive the cannula 16 of the catheter assembly 10 therethrough. Therefore, the through hole 120 may be sized and shaped corresponding to the cannula 16 and the injection site 18. In some embodiments, the cannula 16 is slidably received along a length 117 of the slit 116 and received at least partially within the through hole 120 of the base 110.

In the illustrated embodiment of FIG. 2, the catheter securement device 100 further includes a closure tab 130 fixedly attached to the base 110 and extending from the first major surface 112 of the base 110. The closure tab 130 may be fixedly attached to the base 110 by any suitable method. For example, in some embodiments, the closure tab 130 may be fixedly attached to the base 110 by a suitable adhesive, such as a permanent adhesive. In some embodiments, the closure tab 130 may be fixedly attached to the base 110 by being integrally formed with the base 110. Moreover, in some embodiments, the closure tab 130 may be fixedly attached to the base 110 by stitching.

In some embodiments, the closure tab 130 is configured to be detachably attached to the first major surface 112 of the base 110. The closure tab 130 may be detachably attached to the first major surface 112 by a hook and loop material, such as VELCRO®, an adhesive tape, and the like. In some embodiments, the closure tab 130 may be removably attached to the first major surface 112 by a suitable adhesive, such as a pressure-sensitive adhesive. In some embodiments, the closure tab 130 is configured to be detachably attached to the first major surface 112 of the base 110 in order to at least partially cover and close the slit 116. In some embodiments, the closure tab 130 may be further configured to at least partially cover and close the through hole 120.

In the illustrated embodiment of FIG. 2, the catheter securement device 100 further includes at least one pull tab 136 extending from the base 110. In the illustrated embodiment of FIG. 2, the at least one pull tab 136 is adhesive free, such that the at least one pull tab 136 is detached from the skin 20 of the user 22. In other words, in the illustrated embodiment of FIG. 2, the at least one pull tab 136 is not adhered to the skin 20 of the user 22. Thus, the at least one pull tab 136 may be grippable. Further, in some embodiments, for removal of the base 110 from the skin 20 of the user 22, the base 110 may be stretched by gripping the at least one pull tab 136.

In the illustrated embodiment of FIG. 2, the at least one pull tab 136 includes a pair of opposing pull tabs 136. Thus, for removal of the base 110 from the skin 20 of the user 22, the base 110 may be stretched by gripping the pair of opposing pull tabs 136. The pair of opposing pull tabs 136 may facilitate removal of the base 110 from the skin 20 of the user 22.

The catheter securement device 100 further includes a plurality of lock tabs 140 fixedly attached to the base 110 and extending from the first major surface 112 of the base 110. The lock tabs 140 are spaced apart from each other. The plurality of lock tabs 140 may be fixedly attached to the base 110 by any suitable method. For example, in some embodiments, the plurality of lock tabs 140 may be fixedly attached to the base 110 by a suitable adhesive, such as a permanent adhesive. In some embodiments, the plurality of lock tabs 140 may be fixedly attached to the base 110 by being integrally formed with the base 110. Moreover, in some embodiments, the plurality of lock tabs 140 may be fixedly attached to the base 110 by stitching.

The catheter securement device 100 further includes a housing 160 configured to be placed on the first major surface 112 of the base 110, such that the housing 160 and the first major surface 112 of the base 110 at least partially enclose the catheter assembly 10 therebetween. Specifically, in the illustrated embodiment of FIG. 2, the housing 160 is configured to be placed on the first major surface 112 of the base 110, such that the housing 160 and the first major surface 112 of the base 110 substantially enclose the catheter hub 12 of the catheter assembly 10 therebetween.

Thus, the housing 160 and the base 110 may have any suitable shape to least partially enclose the catheter assembly 10 therebetween. In other words, a shape of the housing 160 and the base 110 may depend upon the catheter assembly 10. Further, the housing 160 may be made of any suitable material, for example, plastic, such as Polyethylene Terephthalate (PET), High-Density Polyethylene (HDPE), Polyvinyl Chloride (PVC), Polypropylene (PP), and the like. In some embodiments, the housing 160 be made of polyimide, polycarbonate, polyurethane, and the like.

In the illustrated embodiment of FIG. 2, the housing 160 further includes one or more first openings 170 corresponding to the one or more lumens 14 of the catheter assembly 10. Each first opening 170 from the one or more first openings 170 is configured to receive a corresponding lumen 14 from the one or more lumens 14 therethrough. In the illustrated embodiment of FIG. 2, the housing 160 further includes a second opening 172 configured to receive the cannula 16 of the catheter assembly 10 therethrough.

The housing 160 further includes a plurality of lock handles 162. Each lock handle 162 from the plurality of lock handles 162 defines an aperture 164 therethrough. Furthermore, each lock tab 140 is configured to be received through the aperture 164 of a corresponding lock handle 162 from the plurality of lock handles 162. Upon being received through the aperture 164 of the corresponding lock handle 162, each lock tab 140 is configured to be detachably attached to the first major surface 112 of the base 110, such that the housing 160 is removably secured to the base 110. In some embodiments, each lock tab 140 may be detachably attached to the first major surface 112 by a hook and loop material, such as VELCRO®, an adhesive tape, and the like. In some embodiments, each lock tab 140 may be detachably attached to the first major surface 112 by a suitable adhesive, such as a pressure-sensitive adhesive.

In the illustrated embodiment of FIG. 2, the plurality of lock handles 162 includes a pair of opposing lock handles 162. Moreover, in the illustrated embodiment of FIG. 2, the plurality of lock tabs 140 includes a pair of opposing lock tabs 140. In the illustrated embodiment of FIG. 2, each lock handle 162 from the pair of opposing lock handles 162 is aligned with a corresponding lock tab 140 from the pair of opposing lock tabs 140. Specifically, in the illustrated embodiment of FIG. 2, each aperture 164 from the pair of opposing lock handles 162 is aligned with the corresponding lock tab 140 from the pair of opposing lock tabs 140, such that the corresponding lock tab 140 is receivable through the aperture 164 of the corresponding lock handle 162 during placement of the housing 160 on the first major surface 112 of the base 110.

In the illustrated embodiment of FIG. 2, the housing 160 further includes one or more stabilizer lugs 168 configured to engage with the catheter assembly 10 in order to inhibit the movement of the catheter assembly 10 relative to the housing 160. In some embodiments, one or more stabilizer lugs 168 may be configured to be received into the one or more holes 13 of the catheter hub 12. In the illustrated embodiment of FIG. 2, two of the stabilizer lugs 168 are configured to be at least partially received into the corresponding two holes 13 of the catheter hub 12.

Therefore, the catheter securement device 100 may secure the catheter assembly 10 firmly onto the skin 20 of the user 22. Furthermore, the catheter securement device 100 may inhibit movement of the catheter assembly 10. Consequently, the catheter securement device 100 may prevent various complications that may occur due to micro-movement of the catheter assembly 10. The catheter securement device 100 may further prevent infection at the injection site 18. Moreover, the catheter securement device 100 may provide improved comfort to the user 22 during use.

FIG. 3 illustrates a schematic cross-sectional view of a portion of the catheter securement device 100 according to an embodiment of the present disclosure.

As discussed above, the base 110 includes the first major surface 112 and the second major surface 114 opposite to the first major surface 112.

Referring to FIGS. 2 and 3, the base 110 has a thickness T1 defined between the first major surface 112 and the second major surface 114. The thickness T1 of the base 110 may depend upon desired application attributes. For example, the thickness T1 of the base 110 may depend upon a design and/or shape of the base 110 to ensure integrity and handleability of the base 110. In some cases, the thickness T1 being lower than about 10 micrometers may not be suitable for strong adhesives. Further, in some cases, the thickness T1 being greater than about 1000 micrometers may require greater stretching forces for removal of the base 110 from the skin 20 than desired. Therefore, in some embodiments, the thickness T1 of the base 110 may range from about 10 micrometers to about 1000 micrometers.

In the illustrated embodiment of FIG. 3, the catheter securement device 100 further includes a base adhesive layer 124 disposed on the second major surface 114 of the base 110. In some embodiments, the base adhesive layer 124 is configured to removably secure the base 110 to the skin 20 of the user 22. In the illustrated embodiment of FIG. 3, the base adhesive layer 124 has a thickness T2. In some embodiments, the thickness T2 of the base adhesive layer 124 may range from about 10 micrometers to about 400 micrometers.

In some embodiments, the base adhesive layer 124 includes a stretch-release adhesive. In some cases, an adhesive strength of the stretch-release adhesive may be less than a cohesive strength of the stretch-release adhesive, such that upon stretching of the base 110, adhesion capabilities of the stretch-release adhesive may essentially disappear. A tack of the stretch-release adhesive may be lost upon stretching of the base 110. Therefore, in some embodiments, the base 110 may be easily removed from the skin 20 of the user 22 by stretching the base 110 by gripping the at least one pull tab 136.

In the illustrated embodiment of FIG. 3, the catheter securement device 100 further includes a release liner 126 disposed on the base adhesive layer 124. The release liner 126 may protect the base adhesive layer 124 from contaminants, such as dust, debris, and the like, prior to use of the catheter securement device 100. The release liner 126 may be peeled off from the base adhesive layer 124 prior to removable securement of the base 110 to the skin 20 of the user 22. The release liner 126 may include any suitable material, such as Polyethylene Terephthalate (PET), High-Density Polyethylene (HDPE), Polyvinyl Chloride (PVC), Polypropylene (PP), and the like.

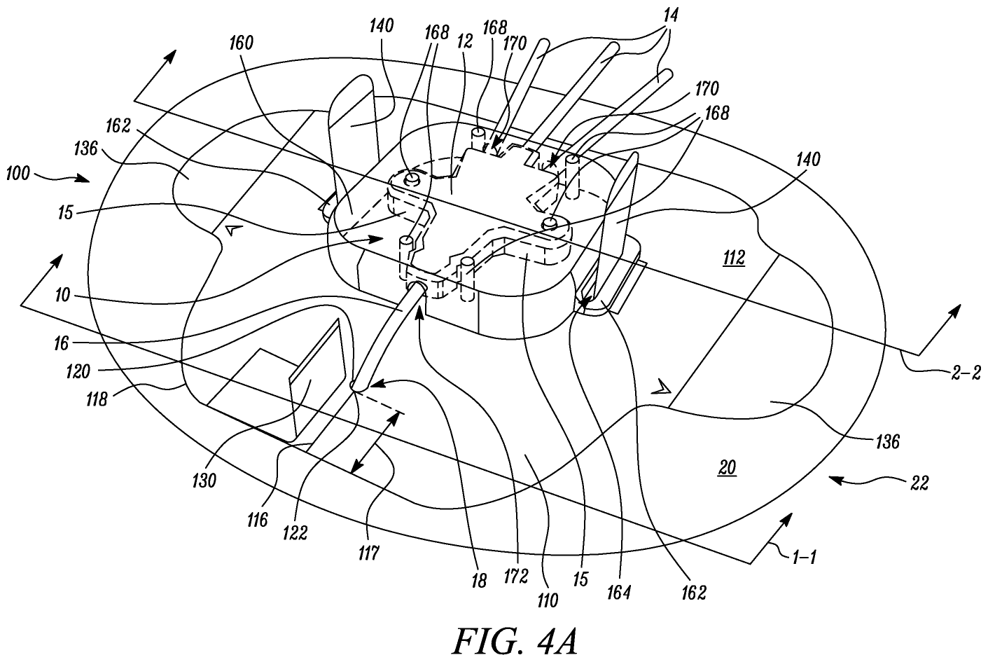

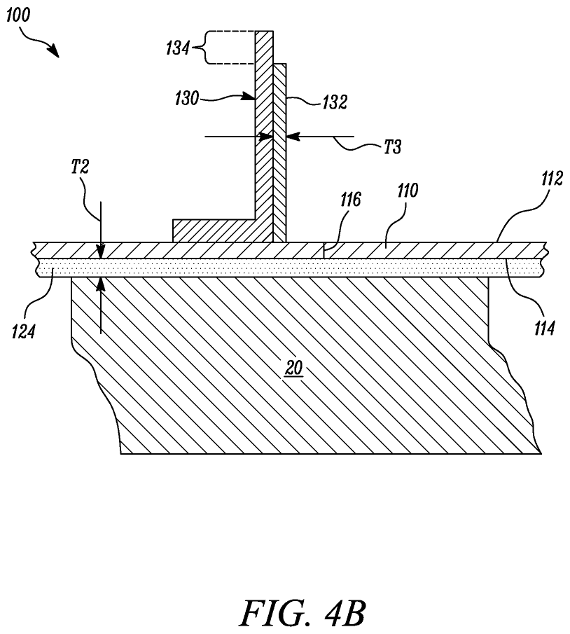

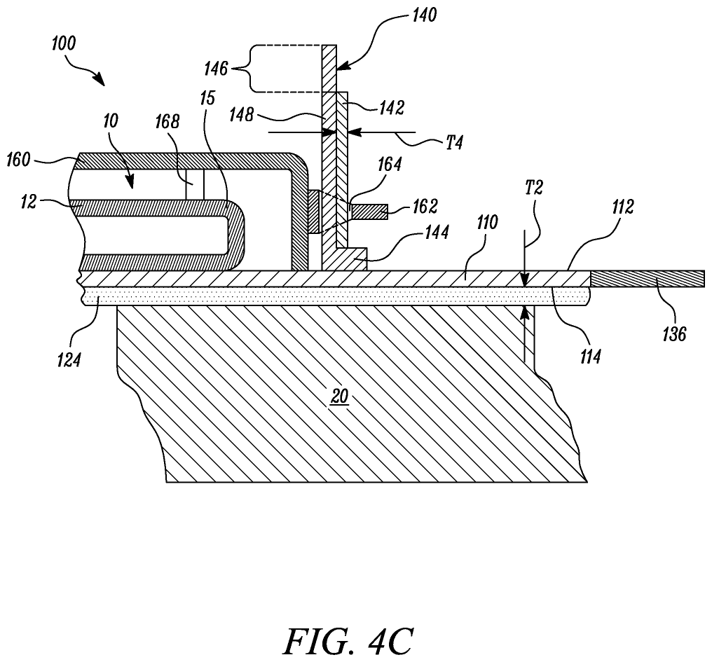

FIG. 4A illustrates a schematic perspective view of the catheter securement device 100 according to an embodiment of the present disclosure.

In the illustrated embodiment of FIG. 4A, the housing 160 is placed on the first major surface 112 of the base 110, such that the housing 160 and the first major surface 112 of the base 110 at least partially enclose the catheter assembly 10 therebetween. Specifically, in the illustrated embodiment of FIG. 4A, the housing 160 is placed on the first major surface 112 of the base 110, such that the housing 160 and the first major surface 112 of the base 110 substantially enclose the catheter hub 12 of the catheter assembly 10 therebetween.

In the illustrated embodiment of FIG. 4A, the one or more stabilizer lugs 168 of the housing 160 engage with the catheter assembly 10 in order to inhibit the movement of the catheter assembly 10 relative to the housing 160. Specifically, in the illustrated embodiment of FIG. 4A, two of the one or more stabilizer lugs 168 engage an outer surface of the catheter hub 12, two of the one or more stabilizer lugs 168 are at least partially received in the respective holes 13 of the catheter hub 12, and two of the one or more stabilizer lugs 168 engage the one or more lumens 14.

In the illustrated embodiment of FIG. 4A, each first opening 170 from the one or more first openings 170 receives the corresponding lumen 14 from the one or more lumens 14 therethrough. Furthermore, in the illustrated embodiment of FIG. 4A, the second opening 172 receives the cannula 16 of the catheter assembly 10 therethrough.

Moreover, in the illustrated embodiment of FIG. 4A, each lock tab 140 is received through the aperture 164 of the corresponding lock handle 162. As discussed above, upon being received through the aperture 164 of the corresponding lock handle 162, each lock tab 140 is configured to be detachably attached to the first major surface 112 of the base 110, such that the housing 160 is removably secured to the base 110.

FIG. 4B illustrates a schematic cross-sectional view of a portion of the catheter securement device 100 taken along a line 1-1 of FIG. 4A. In FIG. 4B, a portion of the catheter securement device 100 is shown prior to detachable attachment of the closure tab 130 to the first major surface 112 of the base 110.

Referring to FIGS. 4A and 4B, in the illustrated embodiment of FIG. 4B, the closure tab 130 includes a closure adhesive layer 132 configured to detachably attach the closure tab 130 to the first major surface 112 of the base 110. In some embodiments, the closure adhesive layer 132 may include a pressure-sensitive adhesive. In some embodiments, the closure adhesive layer 132 may include tackified rubber adhesives, such as natural rubber, olefins, silicones, polyisoprene, polybutadiene, polyurethanes, styrene-isoprene-styrene and styrene-butadiene-styrene block copolymers, and other elastomer; and tackified or untackified acrylic adhesives such as copolymers of iso-octyl acrylate and acrylic acid, which may be polymerized by radiation, solution, suspension, or emulsion techniques.

In the illustrated embodiment of FIG. 4B, the closure tab 130 further includes an end portion 134 free of the closure adhesive layer 132. Therefore, the end portion 134 may be interchangeably referred to as “adhesive-free end portion 134”. Thus, the end portion 134 of the closure tab 130 may be grippable. In some embodiments, the closure tab 130 may be detachably attached to the first major surface 112 by gripping the end portion 134 of the closure tab 130 to at least partially cover and close the slit 116. In some embodiments, the closure tab 130 may be detached from the base 110 by gripping the end portion 134 of the closure tab 130 for exposing and opening the slit 116.

Advantageously, the closure tab 130 may at least partially cover the through hole 120 and the injection site 18, thereby protecting the injection site 18 from micro-organisms, such as bacteria. In some embodiments, an anti-microbial agent may be provided at the injection site 18 and the through hole 120 before detachably attaching the closure tab 130 to the first major surface 112 in order to reduce or prevent infection at the injection site 18. In some embodiments, the closure adhesive layer 132 may include the anti-microbial agent. Examples of the anti-microbial agent include, but are not limited to, iodine, hydrogen peroxide, benzalkonium chloride, and aluminum chlorohydrate.

Further, in the illustrated embodiment of FIG. 4B, the closure adhesive layer 132 disposed on the closure tab 130 has a thickness T3. The thickness T3 of the closure adhesive layer 132 may depend on a strength and a type of adhesive of the closure adhesive layer 132.

In the illustrated embodiment of FIG. 4B, the closure adhesive layer 132 is substantially uniformly disposed on the closure tab 130. However, in some other embodiments, the closure adhesive layer 132 may be non-uniformly and discontinuously disposed on the closure tab 130, for example, in a pattern. In other words, the thickness T3 of the closure adhesive layer 132 may vary along a length of the closure tab 130. In some embodiments, the thickness T3 of the closure adhesive layer 132 may be from about 10 micrometers to about 400 micrometers.

FIG. 4C illustrates a schematic cross-sectional view of a portion of the catheter securement device 100, taken along a line 2-2 of FIG. 4A. In FIG. 4C, a portion of the catheter securement device 100 is shown prior to detachable attachment of the corresponding lock tab 140 to the first major surface 112 of the base 110.

Referring to FIGS. 4A and 4C, in the illustrated embodiment of FIG. 4C, each lock tab 140 includes a lock adhesive layer 142 configured to detachably attach the corresponding lock tab 140 from the plurality of lock tabs 140 to the first major surface 112 of the base 110.

In some embodiments, the lock adhesive layer 142 includes a pressure-sensitive adhesive. In some embodiments, the lock adhesive layer 142 may include tackified rubber adhesives, such as natural rubber, olefins, silicones, polyisoprene, polybutadiene, polyurethanes, styrene-isoprene-styrene and styrene-butadiene-styrene block copolymers, and other elastomer; and tackified or untackified acrylic adhesives such as copolymers of iso-octyl acrylate and acrylic acid, which may be polymerized by radiation, solution, suspension, or emulsion techniques.

In the illustrated embodiment of FIG. 4C, each lock tab 140 further includes a proximal end portion 144, a distal end portion 146, and an intermediate portion 148. The proximal end portion 144 is fixedly attached to the base 110. The proximal end portion 144 may be fixedly attached to the base 110 by any suitable method. For example, in some embodiments, the proximal end portion 144 may be fixedly attached to the base 110 by a suitable adhesive, such as a permanent adhesive. In some embodiments, the proximal end portion 144 may be fixedly attached to the base 110 by being integrally formed with the base 110. Moreover, in some embodiments, the proximal end portion 144 may be fixedly attached to the base 110 by stitching.

In the illustrated embodiment of FIG. 4C, the distal end portion 146 is spaced apart from the proximal end portion 144 and free of the lock adhesive layer 142. Therefore, the distal end portion 146 may be interchangeably referred to as “adhesive-free end portion 146”).

Further, in the illustrated embodiment of FIG. 4C, the intermediate portion 148 is disposed between the proximal end portion 144 and the distal end portion 146. Moreover, in the illustrated embodiment of FIG. 4C, the lock adhesive layer 142 is disposed on the intermediate portion 148.

Before removable attachment to the first major surface 112 of the base 110, each lock tab 140 may be substantially L-shaped. Further, the distal end portion 146 of each lock tab 140 may be grippable. In some embodiments, each lock tab 140 may be detachably attached to the first major surface 112 of the base 110 by gripping the distal end portion 146 of each lock tab 140. In some embodiments, each lock tab 140 may be detached from the first major surface 112 of the base 110 by gripping the distal end portion 146 of each lock tab 140.

In the illustrated embodiment of FIG. 4C, the lock adhesive layer 142 has a thickness T4. The thickness T4 of the lock adhesive layer 142 may depend on a strength and a type of adhesive of the lock adhesive layer 142.

In the illustrated embodiment of FIG. 4C, the lock adhesive layer 142 is substantially uniformly disposed on the intermediate portion 148 of the lock tab 140. However, in some other embodiments, the lock adhesive layer 142 may be non-uniformly and discontinuously disposed on the intermediate portion 148 of the lock tab 140, for example, in a pattern. In other words, the thickness T4 of the lock adhesive layer 142 may vary along a length of the intermediate portion 148. In some embodiments, the thickness T4 of the lock adhesive layer 142 may be from about 10 micrometers to about 400 micrometers.

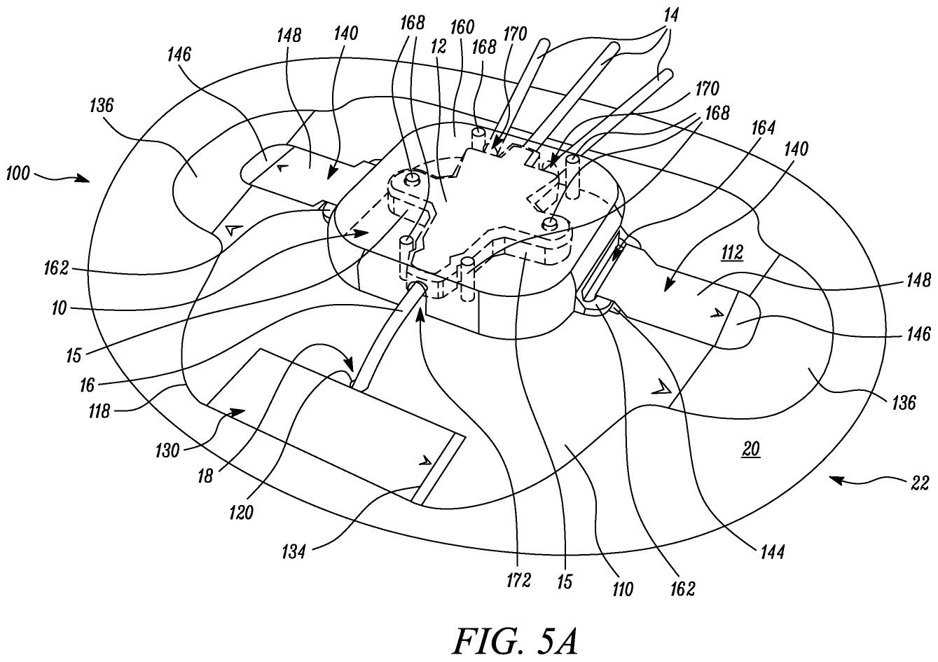

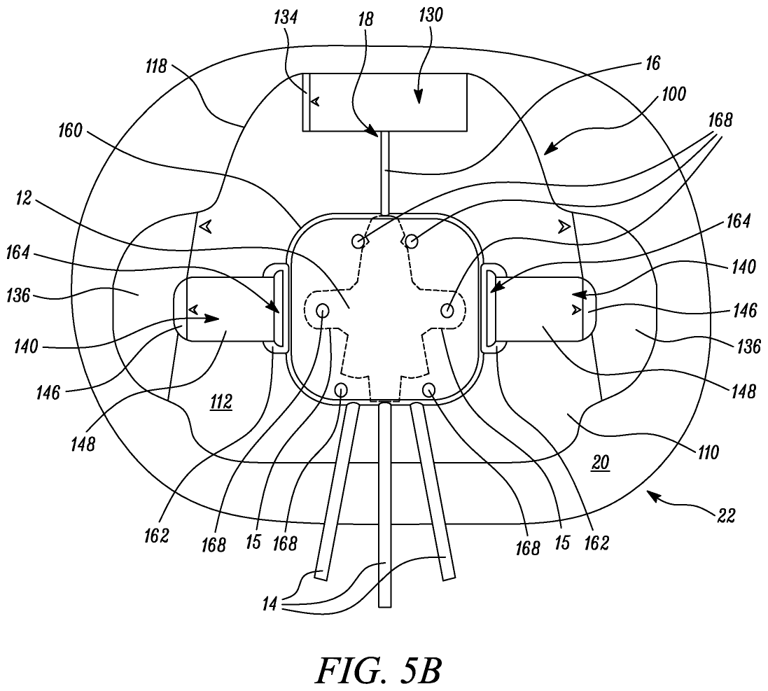

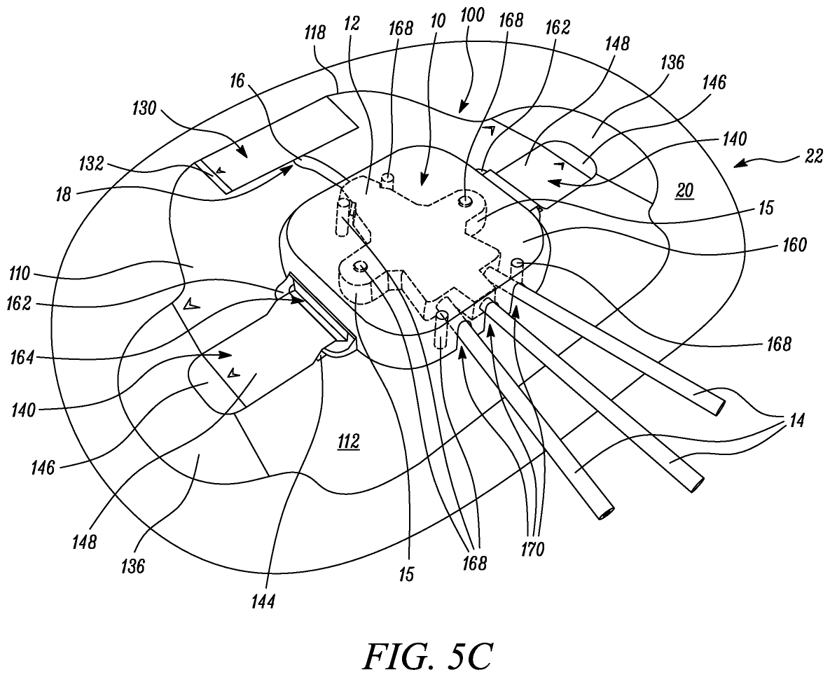

FIGS. 5A, 5B, and 5C illustrate the catheter securement device 100 according to an embodiment of the present disclosure. Specifically, FIG. 5A illustrates a schematic front perspective view of the catheter securement device 100, FIG. 5B illustrates a schematic top view of the catheter securement device 100, and FIG. 5C illustrates a schematic rear perspective view of the catheter securement device 100.

In the illustrated embodiments of FIGS. 5A, 5B, and 5C, the closure tab 130 is detachably attached to the first major surface 112 of the base 110. Specifically, in the illustrated embodiment of FIGS. 5A, 5B, and 5C, the closure adhesive layer 132 (shown in FIG. 4B) of the closure tab 130 detachably attaches the closure tab 130 to the first major surface 112 of the base 110. Therefore, in the illustrated embodiment of FIGS. 5A, 5B, and 5C, the closure tab 130 at least partially covers and closes the slit 116 (shown in FIG. 4A). Further, in the illustrated embodiment of FIGS. 5A, 5B, and 5C, the closure tab 130 at least partially covers and closes the through hole 120.

In the illustrated embodiment of FIGS. 5A, 5B, and 5C, each lock tab 140 from the plurality of lock tabs 140 is attached to the first major surface 112 of the base 110. Specifically, in the illustrated embodiment of FIGS. 5A, 5B, and 5C, the lock adhesive layer 142 (shown in FIG. 4C) of the corresponding lock tab 140 from the plurality of lock tabs 140 attaches the corresponding lock tab 140 to the first major surface 112 of the base 110.

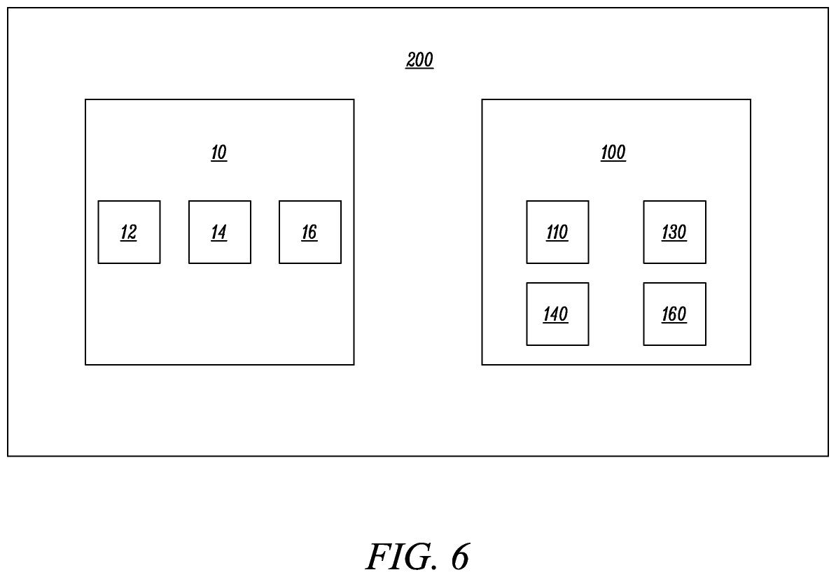

FIG. 6 illustrates a schematic block diagram of a kit 200 according to an embodiment of the present disclosure.

Referring to FIGS. 5A-5C and 6, the kit 200 includes the catheter assembly 10. The kit 200 further includes the catheter securement device 100 for securing the catheter assembly 10 to the skin 20 of the user 22.

In the illustrated embodiment of FIG. 6, the catheter assembly 10 includes the catheter hub 12 disposed on the first major surface 112 of the base 110, the one or more lumens 14 at least partially received within the catheter hub 12, and the cannula 16 at least partially received within the catheter hub 12.

Moreover, the catheter securement device 100 includes the base 110, the plurality of lock tabs 140, and the housing 160. In some embodiments, the catheter securement device 100 further includes the closure tab 130.

In some embodiments, the kit 200 may further include gloves. In some embodiments, the kit 200 may further include a sterilizing material. In some embodiments, the kit 200 may further include a cloth or other absorbent material. In some embodiments, the kit 200 may further an anti-microbial agent. The anti-microbial agent may be in a form of liquid or a gel. In some embodiments, the kit 200 may further include cleaning articles, such as cleaning cloth, cotton balls, cotton swabs, and the like.

The kit 200 may be available to a clinician for use in a sterilized package. The clinician may remove the release liner 126 (shown in FIG. 3) and detachably attach the catheter securement device 100 onto the skin 20. The catheter assembly 10 may therefore be secured and stabilized. In some cases, the clinician may further apply a window dressing over the injection site 18 to provide extra protection from infection at the injection site 18. The slit 116 (shown in FIG. 4A) and the through hole 120 may allow the catheter securement device 100 to be applied to a cannula (e.g., the cannula 16) already inserted into an injection site (e.g., the injection site 18 in FIG. 4A). Further, the closure tab 130 may close the slit 116 after slidably receiving the cannula 16.

The housing 160 of the catheter securement device 100 may allow the catheter assembly 10 to be inspected whenever desired. For example, the housing 160 may be made of a transparent material to enable inspection of the catheter assembly 10. Further, the catheter securement device 100 may allow the clinician to change a position of the catheter securement device 100 without removal of the catheter assembly 10 from the user 22.

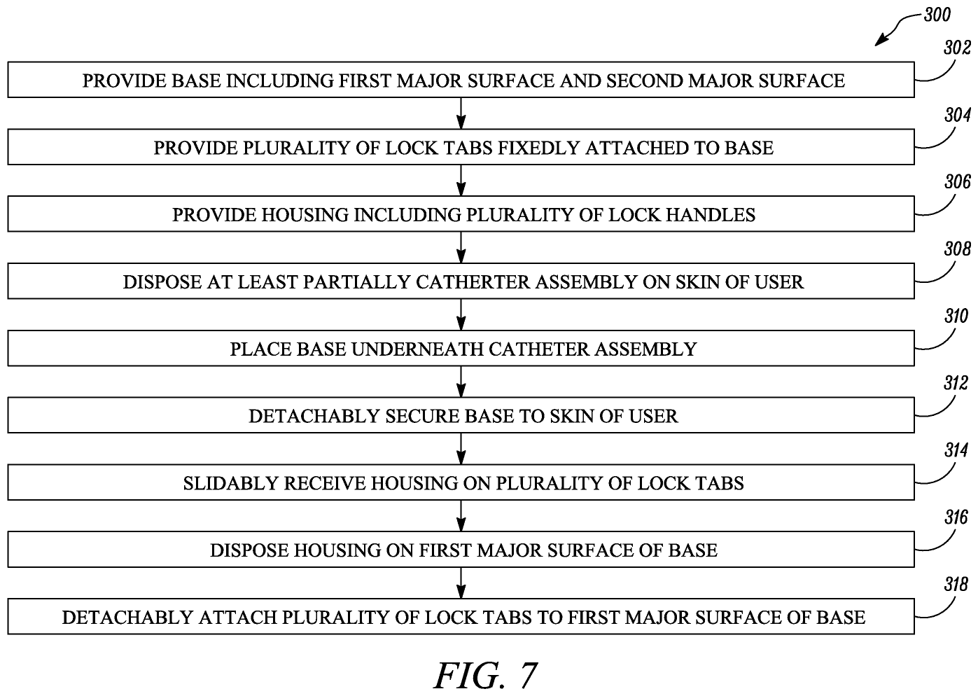

FIG. 7 illustrates a flowchart of a method 300 for use with the catheter assembly 10 (shown in FIG. 1) according to an embodiment of the present disclosure. The method 300 will be described with reference to the catheter assembly 10 of FIG. 1 and the catheter securement device 100 of FIGS. 2-5C.

At step 302, the method 300 includes providing the base 110 including the first major surface 112 and the second major surface 114 opposite to the first major surface 112.

At step 304, the method 300 further includes providing the plurality of lock tabs 140 fixedly attached to the base 110 and extending from the first major surface 112 of the base 110.

At step 306, the method 300 further includes providing the housing 160 including the plurality of lock handles 162. Each lock handle 162 from the plurality of lock handles 162 defines the aperture 164 therethrough.

At step 308, the method 300 further includes disposing at least partially the catheter assembly 10 on the skin 20 of the user 22. In some embodiments, disposing at least partially the catheter assembly 10 on the skin 20 of the user 22 further includes inserting the cannula 16 of the catheter assembly 10 into the injection site 18. In other words, in some embodiments, disposing at least partially the catheter assembly 10 on the skin 20 of the user 22 further includes inserting the cannula 16 of the catheter assembly 10 into the skin 20 at the injection site 18.

At step 310, the method 300 further includes placing the base 110 underneath the catheter assembly 10, such that the first major surface 112 of the base 110 at least partially receives the catheter assembly 10 thereon and the second major surface 114 faces the skin 20 of the user 22.

In some embodiments, placing the base 110 underneath the catheter assembly 10 further includes receiving the catheter hub 12 of the catheter assembly 10 on the first major surface 112 of the base 110. In some embodiments, placing the base 110 underneath the catheter assembly 10 further includes sliding the base 110 underneath the catheter assembly 10, such that the cannula 16 is slidably received along the length 117 of the slit 116 and received at least partially within the through hole 120 of the base 110. In some embodiments, the slit 116 extends from the perimeter 118 of the base to the through hole 120.

At step 312, the method 300 further includes detachably securing the base 110 to the skin 20 of the user 22, such that the second major surface 114 faces the skin 20 of the user 22.

In some embodiments, the method 300 further includes detachably attaching, by the closure adhesive layer 132, the closure tab 130 to the first major surface 112 of the base 110 in order to at least partially cover and close the slit 116 upon receiving the cannula 16 within the through hole 120.

At step 314, the method 300 further includes slidably receiving the housing 160 on the plurality of lock tabs 140, such that each lock tab 140 is received through the aperture 164 of the corresponding lock handle 162 from the plurality of lock handles 162.

At step 316, the method 300 further includes disposing the housing 160 on the first major surface 112 of the base 110, such that the catheter assembly 10 is at least partially enclosed between the first major surface 112 of the base 110 and the housing 160.

In some embodiments, the method 300 further includes receiving the one or more lumens 14 of the catheter assembly 10 through the corresponding one or more first openings 170 of the housing 160. In some embodiments, the method 300 further includes receiving the cannula 16 of the catheter assembly 10 through the second opening 172 of the housing 160.

In some embodiments, the method 300 further includes engaging the one or more stabilizer lugs 168 of the housing 160 with the catheter assembly 10 in order to inhibit the movement of the catheter assembly 10 relative to the housing 160.

At step 318, the method 300 further includes detachably attaching the plurality of lock tabs 140 to the first major surface 112 of the base 110, such that the housing 160 is detachably secured to the base 110. In some embodiments, the plurality of lock tabs 140 is detachably attached to the first major surface 112 of the base 110 by the plurality of corresponding lock adhesive layers 142.

In some embodiments, the method 300 further includes detaching the plurality of lock tabs 140 from the first major surface 112 of the housing 160 by gripping the corresponding adhesive-free end portions 146 of the plurality of lock tabs 140.

In some embodiments, the method 300 further includes detaching the closure tab 130 from the first major surface 112 of the base 110 by gripping the adhesive-free end portion 134 of the closure tab 130.

In some embodiments, the method 300 further includes slidably removing the base 110 from underneath the catheter assembly 10 in order to remove the cannula 16 from the through hole 120 and the slit 116.

In some embodiments, the method 300 further includes detaching the plurality of lock tabs 140 from the first major surface 112 of the base 110. In some embodiments, the method 300 further includes slidably removing the housing 160 from the plurality of lock tabs 140 in order to detach the housing 160 from the base 110. In some embodiments, the method 300 further includes detaching the base 110 from the skin 20 of the user 22. In some embodiments, the method 300 further includes removing the base 110 from underneath the catheter assembly 10.

The catheter securement device 100 and the method 300 of the present disclosure may firmly secure the catheter assembly 10 to the skin 20 of the user 22. Thus, the catheter securement device 100 and the method 300 may prevent dislodgement and accidental removal of the catheter assembly 10 from the skin 20 of the user 22. Further, the catheter securement device 100 and the method 300 may prevent various complications, such as phlebitis, extravasation/infiltration, leakage, occlusion, and bloodstream infections that may occur due to movement of the catheter assembly 10.

Therefore, the catheter securement device 100 and the method 300 may firmly secure the catheter assembly 10 to the skin 20 of the user 22, may inhibit undesirable movement of the catheter assembly 10, may prevent infection at the injection site 18, and may facilitate removal of the catheter securement device 100 after use.

The catheter securement device 100 may also be removably secured to a previously installed catheter assembly. Therefore, the catheter assembly 10 and the catheter securement device 100 can be used by different medical professionals at different times.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A catheter securement device for use with a catheter assembly, the catheter securement device comprising:
a base configured to be removably secured to a skin of a user, wherein the base comprises a first major surface and a second major surface opposite to the first major surface, wherein the first major surface is configured to at least partially receive the catheter assembly thereon, and wherein the second major surface is configured to face the skin of the user;
a plurality of lock tabs fixedly attached to the base and extending from the first major surface of the base, wherein the lock tabs are spaced apart from each other; and
a housing configured to be placed on the first major surface of the base, such that the housing and the first major surface of the base at least partially enclose the catheter assembly therebetween, wherein the housing comprises a plurality of lock handles, and wherein each lock handle from the plurality of lock handles defines an aperture therethrough;
wherein each lock tab is configured to be received through the aperture of a corresponding lock handle from the plurality of lock handles, and wherein, upon being received through the aperture of the corresponding lock handle, each lock tab is configured to be detachably attached to the first major surface of the base, such that the housing is removably secured to the base.

2. The catheter securement device of claim 1, wherein each lock tab comprises a lock adhesive layer configured to detachably attach a corresponding lock tab from the plurality of lock tabs to the first major surface of the base.

3. The catheter securement device of claim 2, wherein each lock tab further comprises:
a proximal end portion fixedly attached to the base;
a distal end portion spaced apart from the proximal end portion and free of the lock adhesive layer; and
an intermediate portion disposed between the proximal end portion and the distal end portion, wherein the lock adhesive layer is disposed on the intermediate portion.

4. The catheter securement device of claim 2, wherein the lock adhesive layer comprises a pressure-sensitive adhesive.

5. The catheter securement device of claim 1, wherein the housing further comprises one or more stabilizer lugs configured to engage with the catheter assembly in order to inhibit a movement of the catheter assembly relative to the housing.

6. The catheter securement device of claim 1, wherein the base further comprises at least one of a polymeric film, a polymeric foam, a polymeric hydrocolloid, and a polymeric alginate.

7. The catheter securement device of claim 1, wherein the housing further comprises one or more first openings corresponding to one or more lumens of the catheter assembly, and wherein each first opening from the one or more first openings is configured to receive a corresponding lumen from the one or more lumens therethrough.

8. The catheter securement device of claim 1, wherein the housing further comprises a second opening configured to receive a cannula of the catheter assembly therethrough.

9. The catheter securement device of claim 8, wherein the base further defines a slit extending from a perimeter of the base and a through hole at an end of the slit distal to the perimeter of the base, wherein the through hole is configured to receive the cannula of the catheter assembly therethrough.

10. The catheter securement device of claim 9, further comprising a closure tab fixedly attached to the base and extending from the first major surface of the base, wherein the closure tab is configured to be detachably attached to the first major surface of the base in order to at least partially cover and close the slit.

11-16. (canceled)

17. A kit comprising: a catheter assembly; and
a catheter securement device for securing the catheter assembly to a skin of a user, the catheter securement device comprising:
a base configured to be removably secured to the skin of the user, wherein the base comprises a first major surface and a second major surface opposite to the first major surface, wherein the first major surface is configured to at least partially receive the catheter assembly thereon, and wherein the second major surface is configured to face the skin of the user;
a plurality of lock tabs fixedly attached to the base and extending from the first major surface of the base, wherein the lock tabs are spaced apart from each other; and
a housing configured to be placed on the first major surface of the base, such that the housing and the first major surface of the base at least partially enclose the catheter assembly therebetween, wherein the housing comprises a plurality of lock handles, and wherein each lock handle from the plurality of lock handles defines an aperture therethrough;
wherein each lock tab is configured to be received through the aperture of a corresponding lock handle from the plurality of lock handles, and wherein, upon being received through the aperture of the corresponding lock handle, each lock tab is configured to be detachably attached to the first major surface of the base, such that the housing is removably secured to the base.

18. The kit of claim 17, wherein each lock tab comprises a lock adhesive layer configured to detachably attach a corresponding lock tab from the plurality of lock tabs to the first major surface of the base.

19. The kit of claim 18, wherein each lock tab further comprises:
a proximal end portion fixedly attached to the base;
a distal end portion spaced apart from the proximal end portion and free of the lock adhesive layer; and
an intermediate portion disposed between the proximal end portion and the distal end portion, wherein the lock adhesive layer is disposed on the intermediate portion.

20-33. (canceled)

34. A method for use with a catheter assembly, the method comprising:
providing a base comprising a first major surface and a second major surface opposite to the first major surface;
providing a plurality of lock tabs fixedly attached to the base and extending from the first major surface of the base;
providing a housing comprising a plurality of lock handles, wherein each lock handle from the plurality of lock handles defines an aperture therethrough;
disposing at least partially the catheter assembly on a skin of a user;
placing the base underneath the catheter assembly, such that the first major surface of the base at least partially receives the catheter assembly thereon and the second major surface faces the skin of the user;
detachably securing the base to the skin of the user, such that the second major surface faces the skin of the user;
slidably receiving the housing on the plurality of lock tabs, such that each lock tab is received through the aperture of a corresponding lock handle from the plurality of lock handles;
disposing the housing on the first major surface of the base, such that the catheter assembly is at least partially enclosed between the first major surface of the base and the housing; and
detachably attaching the plurality of lock tabs to the first major surface of the base, such that the housing is detachably secured to the base.

35. The method of claim 34, wherein disposing at least partially the catheter assembly on the skin of the user further comprises inserting a cannula of the catheter assembly into an injection site.

36. The method of claim 34, wherein placing the base underneath the catheter assembly further comprises receiving a catheter hub of the catheter assembly on the first major surface of the base.

37. The method of claim 34, wherein the plurality of lock tabs is detachably attached to the first major surface of the base by a plurality of corresponding lock adhesive layers.

38. The method of claim 34, further comprising detaching the plurality of lock tabs from the first major surface of the housing by gripping corresponding adhesive-free end portions of the plurality of lock tabs.

39. The method of claim 34, further comprising engaging one or more stabilizer lugs of the housing with the catheter assembly in order to inhibit a movement of the catheter assembly relative to the housing.

40. The method of claim 34, further comprising receiving one or more lumens of the catheter assembly through corresponding one or more first openings of the housing.

41-46. (canceled)
